(19) United States
(12) Patent Application Publication
(10) Pub. No.: US 2012/0171239 A1
(43) Pub. Date: Jul. 5, 2012

(54) Immunogenic Conjugates for Producing Immune Responses to Drugs of Abuse and Methods of Use

(76) Inventor: Steven B. Mizel, Lewisville, NC (US)

(21) Appl. No.: 13/393,145

(22) PCT Filed: Sep. 2, 2010
(86) PCT No.: PCT/US10/47635

§ 371 Date: Mar. 22, 2012

Related U.S. Application Data
(60) Provisional application No. 61/239,617, filed on Sep. 3, 2009.

(51) Int. Cl. A61K 39/00; C07K 1/107; A61P 37/04; A61P 25/36; A61P 25/34; C07K 14/195; A61P 25/30

(52) U.S. Cl. 424/193.1; 530/405; 530/406

(57) ABSTRACT

The present invention is based, in part, on flagellin adjuvants that enhance immune responses directed against drugs of abuse. Provided are conjugates comprising a flagellin adjuvant covalently linked to a drug of abuse or an immunologically similar derivative thereof. Also provided are methods of making the conjugates of the invention and use thereof for administration to a subject, e.g., to produce an immune response in the subject against the drug of abuse, to prevent addiction to the drug of abuse in the subject, to reduce the effect of the drug of abuse in the subject, to reduce the level of the drug of abuse in the brain of the subject, and/or to reduce the addiction in the subject to the drug of abuse.

### RELATED APPLICATION INFORMATION

This application claims the benefit of U.S. Provisional Application No. 61/239,617; Filed Sep. 3, 2009, the disclosure of which is incorporated by reference herein in its entirety.

### STATEMENT OF FEDERAL SUPPORT

This invention was supported in part by funding provided under Grant No. RC2DA028906 from the National Institutes of Health. The United States government has certain rights in this invention.

### FIELD OF THE INVENTION

The present invention relates to immunogenic compositions for inducing an immune response against a drug of abuse and methods of using the same to induce an immune response in a subject, e.g., to prevent or treat drug addiction.

### BACKGROUND OF THE INVENTION

Illicit drug use is not only a major global health problem, but has also had a profoundly negative social, political, and economic impact that continues to increase in magnitude. For example, it is estimated that one out of four Americans between the ages of 26 and 34 have used cocaine in their lifetime and at least one-third of all federal and state prisoners involved in property crimes indicated that they acted to obtain money for drugs (Orson et al., (2008) Ann. N.Y. Acad. Sci. 1141:257-269). Recent estimates suggest that 22-25 million Americans have tried cocaine at least once. There is an alarming increase in the use of illicit drugs such as cocaine and methamphetamine among young people with use estimates in the range of 5-8%. Although treatments such as the nicotine patch or methadone have been useful in reducing abuses to smoking and opiate abuse, there are currently no such treatments for cocaine or methamphetamine abuse. Furthermore, the drug substitution approach requires a high level of patient compliance.

In view of the magnitude of the health problem and the lack of an appropriate drug substitution therapy for addicts, research efforts have focused on the concept of developing vaccines against drugs of abuse. The hypothesis that drives this line of study is quite simple—by reducing the concentration of free drug in plasma via antibody binding, the ability of the drug to reach the brain in sufficient amounts to elicit a pleasurable, reinforcing effect is dramatically reduced. This approach was first used by Bonese et al. to alter the response to heroin in rhesus monkeys (Bonese et al., (1974) Nature 252:708-710). In 1995, Carrera et al. synthesized a cocaine analog (GNC)-keyhole limpet haemocyanin conjugate and used it to immunize rats (Carrera et al., (1995) Nature 378:727-730). Immunized rats exhibited a significant decrease in cocaine-induced locomotor activity and stereotyped behavior. Subsequent studies by a number of groups have confirmed and extended this basic observation (Orson et al., (2008) Ann. N.Y. Acad. Sci. 1141:257-269; Carrera et al., (1995) Nature 378:727-730; Carrera et al., (2001) Proc. Nat. Acad. Sci. USA 98:1988-1992; Fox et al., (1996) Nature Med. 2:1129-1132; Johnson et al., (2000) Exp. Clin. Psychopharm. 8:163-167; Kantak et al., (2000) Psychopharm. 148:251-262; Koetzner et al., (2001) J. Pharmacol. Exp. Ther. 296:789-796; Kosten et al., (2002) Vaccine 20:1196-1204; and Martell et al., (2005) Biol. Reprod. 58:158-164).

More recently, efforts have moved forward with initial clinical trials in humans. Martell et al. (Martell et al., (2005) Biol. Reprod. 58:158-164) conducted a phase I clinical trial with a vaccine containing a cocaine-cholera toxin B conjugate. In this study, cocaine-dependent subjects were given either four doses of 100 micrograms over 8 weeks or 5 doses of 400 micrograms over 12 weeks. There were no significant adverse reactions and the vaccine appeared to be well-tolerated. Analysis of serum anti-cocaine antibody titers revealed a dose-dependent increase that peaked between weeks 10 and 14 and dropped off significantly by 24 weeks. The limitations of this vaccine were evidenced by 1) the substantial variability in the titers from individual subjects; 2) the relatively low titers that were achieved; and 3) the observation that 89% of the subjects in the 400 microgram group relapsed within 6 months and 43% in the 2000 microgram group.

These results point to some of the major challenges in this area. First and foremost is the need to develop vaccines that elicit dramatically more robust drug-specific serum IgG titers than have previously been obtained. Given the possibility that substance abusers will escalate the amount of material that they use to recapture the desired pharmacological effects, the circulating titers of specific IgG are preferably of sufficient magnitude to provide a substantial buffer to easily accommodate the maximal levels of drug that might be reached. Further, in view of the issues related to patient compliance, the number of immunizations can be minimized to the extent possible. Finally, the amounts of vaccine that have been used to date are relatively high and therefore the costs associated with vaccine production and use is likely to be unacceptably high. If vaccines against drugs of abuse are to become a practical reality, then it would be advantageous to develop new approaches that promote robust antibody responses with far smaller amounts of vaccine and fewer immunizations.

The development of efficacious vaccines against drugs of abuse would not only have a positive impact on reversing abuse but would make such efforts easier to achieve and less costly due to the limited number of interventions (i.e., immunizations) and vaccine amounts required. Given the number of individuals with abuses to one or more substances (in the millions), a successful vaccine program would not only improve the health of these individuals, but would reduce the social, economic, and political costs associated with addictive behavior.

Accordingly, there is a need in the art for improved compositions and methods for immunizing subjects against drugs of abuse, e.g., to prevent or treat drug addiction.

### SUMMARY OF THE INVENTION

The present invention is based, in part, on flagellin adjuvants that enhance immune responses directed against drugs of abuse.

Accordingly, as one aspect, the invention provides a conjugate comprising a flagellin adjuvant (e.g., a recombinant flagellin monomer) covalently linked to a drug of abuse or an immunologically similar derivative thereof.

As another aspect, the invention provides a method of making a conjugate of the invention, wherein the method comprises covalently linking a drug of abuse or immunologically similar derivative thereof to a flagellin adjuvant.

Also provided are compositions comprising a conjugate of the invention, and immunogenic formulations comprising a conjugate of the invention in a pharmaceutically acceptable carrier.

As a further aspect, the invention provides an article of manufacture comprising a closed, pathogen-impermeable container and a sterile preparation enclosed within said container, wherein said preparation comprises an immunogenic formulation of the invention.

As yet a further aspect, the invention provides a method of producing an immune response against a drug of abuse in a mammalian subject, the method comprising administering a conjugate or immunogenic formulation of the invention to the mammalian subject in an amount effective to produce an immune response in the mammalian subject against the drug of abuse.

Still a further aspect of the invention is a method of preventing addiction to a drug of abuse in a mammalian subject, the method comprising administering a conjugate or immunogenic formulation of the invention to the mammalian subject in an amount effective to prevent addiction to the drug of abuse in the mammalian subject.

As another aspect, the invention provides a method of reducing the effect of a drug of abuse in a mammalian subject, the method comprising administering a conjugate or immunogenic formulation of the invention to the mammalian subject in an amount effective to reduce the effect of the drug of abuse in the mammalian subject.

As a further aspect, the invention provides a method of reducing the amount of a drug of abuse in the brain of a mammalian subject, the method comprising administering a conjugate or immunogenic formulation of the invention to the mammalian subject in an amount effective to reduce the amount of the drug of abuse in the brain of the mammalian subject.

As still another aspect, the invention provides a method of treating drug addiction in a mammalian subject, the method comprising administering a conjugate or immunogenic formulation of the invention to a mammalian subject addicted to a drug of abuse in an amount effective to reduce the addiction in the mammalian subject to the drug of abuse.

Also provided is the use of a conjugate of the invention in the manufacture of a medicament to prevent or treat addiction to a drug of abuse in a mammalian subject. Further provided is a conjugate of the invention for the prevention or treatment of addiction to a drug of abuse in a mammalian subject.

These and other aspects of the invention are set forth in more detail in the description of the invention below.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

### I. DEFINITIONS

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Numerical ranges as described herein are intended to be inclusive unless the context indicates otherwise. For example, the numerical range of “1 to 10” or “1-10” is intended to be inclusive of the values 1 and 10.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

As used herein, the transitional phrase “consisting essentially of” is to be interpreted as encompassing the recited materials or steps “and those that do not materially affect the basic and novel characteristic(s)” of the claimed invention (e.g., immunogenic and/or adjuvant activity). See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.” Further, by “consisting essentially of” (and grammatical variants) as used herein, it is meant that the indicated polypeptide, conjugate, composition, formulation and the like does not include any other material elements (i.e., elements that materially impact the structure and/or function of the polypeptide, conjugate, nucleic acid, composition or formulation). The term “materially altered,” as applied to polypeptides, conjugates, compositions and immunogenic formulations of the invention, refers to an increase or decrease in immunogenic or adjuvant activity of at least about 15%, 25% or 50% or more as compared with the activity of a polypeptide, conjugate, composition or immunogenic formulation consisting of the recited elements.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

An “isolated” polypeptide indicates that the polypeptide is at least partially purified away from some of the other components of the naturally occurring organism or virus with which it is naturally associated.

The terms “immunogen” and “antigen” are used interchangeably herein and mean any compound (including polypeptides) to which a cellular and/or humoral immune response can be directed.

As used herein, the terms “enhance,” “enhances,” and “enhancing” an immune response (and similar terms), optionally a neutralizing immune response, indicate that the immune response (e.g., antigen-specific IgG production), optionally a neutralizing immune response, is increased by at least about 50%, 2-fold, 3-fold, 4-fold, 5-fold, 10-fold, 15-fold, 20-fold, 30-fold, 40-fold, 50-fold, 60-fold, 75-fold, 100-fold, 150-fold, 500-fold, 1000-fold or more.

As used herein, the terms “reduce,” “reduces,” “reduction” and similar terms mean a decrease of at least about 25%, 35%, 50%, 75%, 80%, 85%, 90%, 95%, 97% or more. In particular embodiments, the reduction results in no or essentially no (i.e., an insignificant amount, e.g., less than about 10% or even 5%) detectible activity.

As used herein, an amino acid sequence that is “substantially identical” or “substantially similar” to a reference amino acid sequence is at least about 75%, 80%, 85%, 90%, 95%, 97%, 98% or 99% identical or similar, respectively, to the reference amino acid sequence.

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), or by inspection.

Another suitable algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al., (1997) Nucleic Acids Res. 25, 3389-3402.

An “effective amount,” as used herein, refers to an amount that imparts a desired effect, which is optionally a therapeutic or prophylactic effect.

By the term “treat,” “treating” or “treatment of” (and grammatical variations thereof) it is meant that the severity of the subject”s condition is reduced, at least partially improved or ameliorated and/or that some alleviation, mitigation or decrease in at least one clinical symptom is achieved and/or there is a delay in the progression of the disease or disorder.

A “treatment effective” amount as used herein is an amount that is sufficient to treat (as defined herein) the subject. Those skilled in the art will appreciate that the therapeutic effects need not be complete or curative, as long as some benefit is provided to the subject.

The terms “prevent,” “preventing” and “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The terms “vaccination” and “immunization” are well-understood in the art, and are used interchangeably herein unless otherwise indicated. For example, the terms vaccination or immunization can be understood to be a process that increases an organism's immune response to antigen. In the case of the present invention, vaccination or immunization against a drug of abuse can be used to prevent and/or treat addiction, e.g., to reduce the effects of a drug of abuse in the subject and/or to reduce the amount or concentration of the drug of abuse and/or an active metabolite thereof in the central nervous system [e.g., brain] of the subject.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

The terms “neutralizing immune response” or “neutralizing antibody response” (and similar terms) as used herein indicates that the immune response or antibody response, respectively, confers some benefit to the subject in that it prevents or reduces the effects of the drug of abuse in the subject and/or reduces the amount or concentration of the drug of abuse (and/or active metabolite) in the central nervous system (e.g., brain), for example, to prevent or treat addiction. The neutralizing immune response or neutralizing antibody response need not provide complete protection or neutralization as long as some benefit is conferred to the subject.

As used herein, the term “effect” or “effects” of a drug of abuse includes any observable effect in the subject, including physiological, psychological and/or behavioral effects, that is induced by the drug of abuse. Exemplary physiological effects include increases or decreases in heart rate, breathing rate, eye dilation, and/or pain reflex. Other physiological effects include elevations in extracellular dopamine, serotonin and/or norepinephrine concentrations in the brain and/or reductions in D₂ binding potential in the brain (e.g., the mesolimbocortical neurons). Examples of psychological effects include changes in mood, e.g., euphoria. Examples of behavioral effects include increases or decreases in locomotor activity, sleep, irritability and/or agitation and/or increases in drug-seeking behavior.

As used herein an “immunologically similar derivative” of a drug of abuse is a molecule (e.g., one that is structurally related to the drug of abuse) that induces an antibody response, where the antibodies recognize and bind to the drug of abuse. In one embodiment, the immunologically similar derivative comprises one or more epitopes identical to an epitope(s) on the drug of abuse. In embodiments of the invention, the antibody preferentially binds to the drug of abuse as compared with inactive metabolites thereof.

### II. Drugs of Abuse

Drugs of abuse according to the present invention include any drug substance that results in physical and/or psychological dependence in the user. For example, physical dependence can result in withdrawal symptoms (e.g., tremors) when the drug is withdrawn. Nonlimiting examples of drugs of abuse include: a cannabinoid (e.g., marijuana or hashish; active ingredient delta-9-tetrahydrocannabinol [THC]); a depressant such as a barbiturate (e.g., Amytal®, Nembutal®, Seconal®, Phenobarbital®), a benzodiazepine (e.g., Ativan®, Halcion®, Librium®, Valium®, Xanax®), flunitrazepam, gamma-hydroxybutyrate and methaqualone; a dissociative anesthetic such as ketamine, phencyclidine (PCP) and phencyclidine derivatives; hallucinogens such as lysergic acid diethylamide (LSD), mescaline and psilocybin; opioids and opium derivatives such as heroin, morphine and morphine derivatives, thebaine, hydromorphone, oxycodone, hydrocodone, codeine, meperidine, and pentazocine; stimulants such as cocaine and cocaine derivatives, amphetamines, methylenedioxy-methamphetamine (MDMA), methamphetamine, methcathinone, methylphenidate and nicotine and nicotine derivatives; fentanyl and fentanyl derivatives; and any combination of the foregoing.

Examples of cocaine derivatives include without limitation: tropane derivatives, (−) cocaine, cocaine propyl ester, RTI-128, RTI-66, RTI-160, RTI-192, m-hydroxycocaine, WIN 35,065-2, WIN 35,428, RTI-31, RTI-32, RTI-55, RTI-111, m-hydroxybenzoylecgonine, p-hydroxybenzoylecgonine, RTI-113, tropine, benztropine, 4′,4″-difluoro-3α-diphenylmethoxytropane, hyoscyamine-N-oxide, methylanisotropine, tropisetronmethiodide, anisodine, scopolamine, scopolamine-N-oxide, methylscopolamine, N-butylscopalamine, (−) pseudococaine, (+) cocaine, norcaine, benzoylnorecgonine, (+) pseudococaine, ecgonidine, exo-6-hydroxytropinone and methylcocaethylene, cocaine derivatives as described in U.S. Pat. No. 6,383,490, and any combination of the foregoing. In embodiments of the invention, the cocaine derivative is not norcocaine and/or benzoylnorecgonine. Tropane derivatives that induce antibodies that block cocaine uptake by the brain are described in the literature (see, e.g., Kinsey et al., (2009) Immunol. Cell Biol. 87: 309-314; and Carrera et al., (2001) Proc. Nat. Acad. Sci. USA 98:1988-1992). Nonlimiting examples of tropane derivatives include TA-CD (a N-succinyl derivative of cocaine) and GND (having an acyl chain linked to the 2-carbon of tropane through an amide linkage, and an amide linkage between the tropane ring and benzyl group to provide additional stability). The structures of cocaine, TA-CD and GND are shown below.

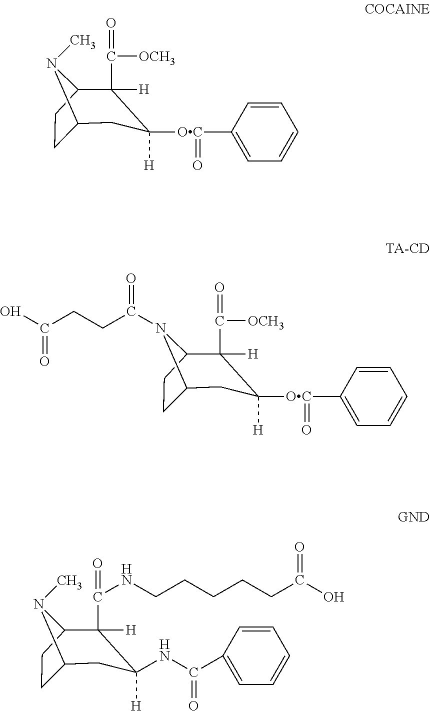

Those skilled in the art will recognize that in embodiments of the invention, it will be advantageous to use an immunologically similar derivative to a drug of abuse to form the conjugates of the invention rather than the drug of abuse itself. For example, many drugs of abuse are metabolized rapidly upon administration, which may be undesirable from the standpoint of immunization. An immunologically similar derivative can be selected that has greater stability in vivo. Further, an immunologically similar derivative to the drug of abuse may facilitate conjugation to the flagellin adjuvant.

In representative embodiments, the immunologically similar derivative can be chosen so as to selectively or preferentially induce an immune response against the drug of abuse (e.g., a high affinity response) as compared with inactive metabolites of the drug of abuse (e.g., a low affinity response or no detectable response). For example, in the case of cocaine, in representative embodiments, the immunologically similar cocaine derivative induces a high affinity antibody response against cocaine, but only a relatively low affinity response or no detectable response against inactive cocaine metabolites such as norcocaine and/or benzoylecognine. Further, with respect to conjugates directed against cocaine, in embodiments of the invention, the problems posed by the rapid degradation of cocaine can be avoided by conjugating flagellin to an immunologically similar cocaine derivative. For example, in embodiments of the invention, the flagellin conjugate can comprise a synthetic tropane derivative that mimics the structure of cocaine (e.g., TA-CD or GND).

Production of a conjugate of the invention may involve modifying the drug sufficiently to render it capable of being conjugated to flagellin while maintaining enough of the structure so that it is recognized in its free state. Methods of modifying compounds to render them suitable for conjugation are well-known in the art, and as is evident from the discussion below, a plethora of well known techniques may be utilized to prepare any given drug and/or derivative thereof for conjugation.

### III. Flagellin Adjuvants

Flagellin proteins are known and described, for example, in U.S. Pat. Nos. 6,585,980, 6130,082; 5,888,810; 5,618,533; 4,886,748 and U.S. Patent Publication No. US 2003/0044429 A1; and Donnelly et al., (2002) J. Biol. Chem. 43: 40456. Most gram-negative bacteria express flagella, which are surface structures that provide motility. The flagella are formed from a basal body, a filament, and a hook that connects the two. The filament is formed of a long polymer of a single protein, flagellin, with a small cap protein at the end. Polymerization of flagellin is mediated by conserved regions at the N- and C-termini, whereas the intervening hypervariable region of the flagellin protein is very diverse in sequence and length among species.

The flagellin adjuvant can be derived from a flagellin from any suitable source. A number of flagellin genes have been cloned and sequenced (see, e.g., Kuwajima et al., (1986) J. Bact. 168:1479; Wei et al., (1985) J. Mol. Biol. 186:791-803; and Gill et al., (1983) J. Biol. Chem. 258:7395-7401). Non-limiting sources of flagellins include but are not limited to S. enteritidis, S. typhimurium, S. dublin, H. pylori, V. cholera, S. marcesens, S. flexneri, S. enterica, T. pallidum, L. pneumophila, B. burgdorferei, C. difficile, A. tumefaciens, R. meliloti, B. clarridgeiae, R. lupine, P. mirabilis, B. subtilis, P. aeruginosa, and E. coli.

The N-terminal and C-terminal constant regions of flagellin are well characterized in the art and have been described, for example, in Mimori-Kiyosue et al., (1997) J. Mol. Virol. 270:222-237; lino et al., (1977) Ann. Rev. Genet. 11:161-182; and Schoenhals et al, (1993) J. Bacteriol. 175:5395-5402. As is understood by those skilled in the art, the size of the constant regions will vary somewhat depending on the source of the flagellin protein. In general, the N-terminal constant domain includes the approximately 170 or 180 N-terminal amino acids of the protein, whereas the C-terminal constant domain typically spans the approximately 85 to 100 C-terminal amino acids. The central hypervariable region varies considerably by size and sequence among bacteria, and accounts for most of the difference in molecular mass. The N- and C-terminal constant regions of flagellin proteins from a variety of bacteria are known, and others can be readily identified by those skilled in the art using known alignment techniques, which are facilitated by the elucidation of the crystal structure of the flagellin monomer (Samatey et al., (2001) Nature 41:331).

The terms “flagellin,” “flagellin N-terminal constant region” and “flagellin C-terminal constant region” include active fragments and modifications of any of the foregoing, which include for example, modifications that enhance the immune response (e.g., a neutralizing immune response) to the drug of abuse (e.g., by activating the TLR5 pathway). As further illustrations, the native flagellin or flagellin regions can be modified to increase safety and/or immune response and/or as a result of cloning procedures or other laboratory manipulations. In some embodiments, the flagellin comprises the full-length flagellin or, alternatively, can comprise an active fragment thereof. Further, the terms “flagellin,” “flagellin N-terminal constant region” and “flagellin C-terminal constant region” and like terms include polypeptides that comprise, consist essentially of, or consist of the naturally occurring amino acid sequences and further encompass polypeptides that comprise, consist essentially of, or consist of an amino acid sequence that is substantially identical or similar to the amino acid sequence of a naturally occurring flagellin, flagellin N-terminal constant region or flagellin C-terminal constant region, respectively, or an active fragment thereof.

Generally, the flagellin adjuvants of the invention are recombinant polypeptides.

As used herein, an “active fragment” of a flagellin, flagellin N-terminal constant region, C-terminal constant region, or any other flagellin region is a fragment of at least about 50, 75, 100, 125, 150, 200, 250 or 300 or more contiguous amino acids and/or less than about 300, 250, 200, 150, 125, 100 or 75 contiguous amino acids, including any combination thereof as long as the lower limit is less than the upper limit, where the active fragment enhances the immune response (e.g., a neutralizing immune response) to the drug of abuse in a host (e.g., by activating the TLR5 pathway). In particular embodiments, the active fragment enhances the immune response (e.g., a neutralizing immune response) to the drug of abuse at least about 50%, 75%, 80%, 85%, 90%, or 95% or more of the level observed with the full-length flagellin or flagellin region, or enhances the immune response to the same or essentially the same extent as the full-length flagellin or flagellin region or enhances the immune response to an even greater extent than the full-length flagellin or flagellin region. Methods of measuring the immune response are well-known in the art (e.g., measurement of antigen-specific IgG). Further, in embodiments of the invention an “active fragment” of a flagellin, flagellin N-terminal constant region, C-terminal constant region, or any other flagellin domain induces an immune response (e.g., a neutralizing immune response) in a host against the drug of abuse (e.g., IgG that react with the drug of abuse), that is at least about 50%, 75%, 80%, 85%, 90%, or 95% or more of the immune response induced by the full-length flagellin or flagellin region, or induces an immune response that is the same as or essentially the same as the full-length flagellin or flagellin region or induces an immune response that is even greater than the immune response induced by the full-length flagellin or flagellin region.

In embodiments of the invention, a “modified” flagellin, flagellin N-terminal constant region, C-terminal constant region, or any other flagellin region (and similar terms) enhances the immune response (e.g., a neutralizing immune response) to the drug of abuse to at least about 50%, 75%, 80%, 85%, 90%, or 95% or more of the level of enhancement observed with the native flagellin or flagellin region, or enhances the immune response to the same or essentially the same extent as the native flagellin or flagellin region or enhances the immune response to an even greater extent than the native flagellin or flagellin region. Methods of measuring the immune response are well-known in the art (e.g., measurement of antigen-specific IgG). Further, in embodiments of the invention a “modified” flagellin, flagellin N-terminal constant region, C-terminal constant region, or any other flagellin region induces an immune response (e.g., a neutralizing immune response) in a host against the drug of abuse (e.g., IgG that react with the drug of abuse), that is at least about 50%, 75%, 80%, 85%, 90%, or 95% or more of the immune response induced by the native flagellin or flagellin region, or induces an immune response that is the same as or essentially the same as the native flagellin or flagellin region or induces an immune response that is even greater than the immune response induced by the native flagellin or flagellin region.

A great deal of structure/function characterization of flagellin proteins has been reported in the literature. Those skilled in the art will be able to identify other suitable flagellin adjuvants within the scope of the present invention, in addition to those specifically disclosed herein, using no more than routine skill. For example, the circulating IgG titers against an antigen following administration of a flagellin fusion protein or flagellin composition (i.e., flagellin+antigen) of the invention can be compared with the circulating IgG induced by administration of the antigen alone.

Generally, the flagellin N-terminal and/or C-terminal constant region comprises a TLR5 recognition site(s) and is able to activate the TLR5 pathway. Regions of the flagellin protein involved in TLR5 signaling have been identified, for example, by Smith et al. (2003) Nat. Immunol. 4:1247-1253 (e.g., amino acids 78-129, 135-173 and 394-444 of S. typhimurium flagellin or orthologs or modified forms thereof). Further, in representative embodiments, the N-terminal constant region comprises the N-terminal RINSA domain (amino acids 31-52 of the S. dublin flagellin) as described by Eaves-Pyles et al. (2001) J. Immunology 167: 7009-7016, or an ortholog or modified form thereof that enhances the immune response to the drug of abuse.

In other embodiments, the N-terminal constant region comprises the D1 and D2 domains, and the C-terminal constant region comprises the D1 and D2 domains (Eaves-Pyles et al. (2001) J. Immunology 167: 7009-7016) or a modified form thereof.

In still further representative embodiments, the flagellin N-terminal and/or C-terminal constant region comprises the peptide GAVQNRFNSAIT (SEC) ID NO:1) as described by U.S. Patent Publication No. US 2003/0044429 A1 to Alderem et al., or an ortholog or modification thereof.

In still other embodiments, the N-terminal constant domain comprises the “motif N” (e.g., amino acids 98-108 of the S. muenchen flagellin) and/or the C-terminal constant domain comprises the “motif C” (e.g., amino acids 441-449 of S. muenchen flagellin) identified by Kanneganti et al., (2004) J. Biol. Chem. 279:5667-5676, or an ortholog or modified form thereof that enhances an immune response (e.g., a neutralizing immune response) to the drug of abuse.

In other illustrative embodiments, the N-terminal constant domain comprises amino acids 88 to 97 of the P. aeruginosa flagellin (see, e.g., Verma et al., (2005) Infect. Immun. 73:8237-8246) or an ortholog or modified form thereof.

In some embodiments of the invention, the flagellin hypervariable region between the constant regions is deleted (in whole or in part); in other embodiments the hypervariable region is present.

Further, the flagellin adjuvant can comprise a hinge region between the N-terminal constant and C-terminal constant regions. The hypervariable region or a S. pneumoniae antigen(s) can function as a hinge region. Additionally, or alternatively, a segment of about 2, 3, 4, 6, 8, 10, 15, 20, 30, 50 or more amino acids can function as a hinge region.

Optionally, the flagellin adjuvant can be a fusion protein comprising any other polypeptide of interest, which can be fused to the N-terminus, C-terminus, and/or be inserted in the hypervariable region, which may be partially or completely deleted. In embodiments of the invention, the flagellin adjuvant is a fusion protein comprising an antigenic polypeptide (e.g., an antigen from a pathogenic organism or virus). For example, the flagellin adjuvant can be a fusion protein, e.g., a fusion protein comprising an immunomodulatory compound. To illustrate, it is known in the art that immune responses can be enhanced by an immunomodulatory cytokine or chemokine (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell growth factor, CD40 Ligand, tumor necrosis factor-α, tumor necrosis factor-β, monocyte chemoattractant protein-1, granulocyte-macrophage colony stimulating factor, lymphotoxin, CCL25 [MECK], and CCL28 [TECH]) or active fragments thereof. In embodiments, the flagellin adjuvant is not a fusion protein or not a fusion protein with an antigenic polypeptide.

### IV. Conjugates

The conjugates of the invention comprise a flagellin adjuvant covalently linked to one or more drugs of abuse or immunologically similar derivatives thereof (e.g., as described above). The covalent linkage can be a direct covalent linkage and/or can comprise cross-linking between the flagellin adjuvant and the drug of abuse or immunologically similar derivative. In representative embodiments, the covalent linkage comprises a linker between the flagellin adjuvant and the drug of abuse or immunologically similar derivative.

In embodiments of the invention, the conjugate comprises two or more (e.g., two, three, four, five or more) different drugs of abuse (or immunologically similar derivatives thereof), for example, cocaine and methamphetamine or immunologically similar derivatives thereof. In embodiments of the invention, the conjugate comprises one drug of abuse or immunologically similar derivative thereof.

In embodiments of the invention, the flagellin adjuvant is a fusion protein comprising any other polypeptide of interest, e.g., an antigenic polypeptide and/or an immunomodulatory polypeptide (as described herein). In other embodiments, the flagellin adjuvant is not a fusion protein comprising a polypeptide of interest (e.g., an antigenic polypeptide and/or an immunomodulatory polypeptide).

Any suitable method can be employed to conjugate the drug of abuse or immunologically similar derivative to the flagellin adjuvant. Methods of conjugating small organic molecules, such as the drug of abuse (or immunologically similar derivatives thereof), to proteins, such as the flagellin adjuvant, are known in the art.

The conjugates can be prepared with any suitable ratio of the flagellin adjuvant to the drug of abuse or immunologically similar derivative, which may be optimized to enhance the immunogenicity of the antigen and/or the adjuvant activity of the flagellin adjuvant. In representative embodiments, a ratio of drug to flagellin adjuvant of greater than about 1:1 is used. In other embodiments, a ratio of drug to flagellin adjuvant of less than about 1:1 is used. In still other embodiments, a ratio of drug to flagellin adjuvant of about 1:1 is used. In representative embodiments, conjugates with a drug to flagellin adjuvant ratio of between about 1:2, 1:3, 1:5, 1:10 or 1:15 or more (excess polypeptide) and about 2:1, 3:1, 4:1, 5:1, 6:1, 7:1, 8:1, 9:1, 10:1 or 15:1 or more (excess drug) are used. The ratio of drug to flagellin can be selected to optimize the robustness of the immune response while retaining TLR5 signaling activity of the flagellin adjuvant. In representative embodiments, the conjugate comprises from about 2 to 12, about 3 to 10, about 4 to 8, optionally about 5, drug molecules (or immunologically similar derivatives thereof) conjugated to each flagellin protein.

Generally, the conjugation is carried out so that the conjugates of the invention are able to induce an immune response to the drug of abuse and, optionally, to the flagellin adjuvant itself. Further, generally the conjugates of the invention are able to activate TLR5 on antigen presenting cells (e.g., dendritic cells). In addition, in embodiments of the invention, the conjugates of the invention are able to induce cellular and/or humoral immune responses. In embodiments of the invention, the conjugates are able to induce innate immunity. In embodiments of the invention, the conjugates of the invention have both mucosal and/or systemic adjuvant activity. In embodiments of the invention, the conjugates are able to activate naïve T cells and/or memory T cells. In embodiments of the invention, the conjugates enhance the interaction of antigen-presenting cells and flagellin-specific naïve and/or memory T cells. In embodiments of the invention, the conjugates enhance interaction of activated flagellin-specific helper T cells and B cells specific to the drug of abuse. In embodiments of the invention, the conjugate is processed via the Class II MHC antigen-processing pathway.

The conjugates of the present invention are prepared by covalently linking (coupling) one or more drugs of abuse or immunologically similar derivatives thereof to flagellin. There are a large number of functional groups that can be used in order to facilitate the linking or conjugation of flagellin to a small molecule, such as a drug of abuse. These include functional moieties such as carboxylic acids, anhydrides, mixed anhydrides, acyl halides, acyl azides, alkyl halides, N-maleimides, imino esters, isocyanates, amines, thiols, and isothiocyanates and others known to the skilled artisan. These moieties are capable of forming a covalent bond with a reactive group of a protein molecule. Depending upon the functional moiety used, the reactive group may be the epsilon amino group of a lysine residue, the N-terminal amino group, a carboxy group or a thiol group. One skilled in the art would recognize that other suitable activating groups and conjugation techniques can be used (see, for example, Wong, CHEMISTRY OF PROTEIN CONJUGATION AND CROSS-LINKING (1991)) (see also Hermanson, BIOCONJUGATE TECHNIQUES (1996);Dick and Beurret, Contrib. Microbiol. Immunol. 10:48-114 (1989)).

In embodiments of the invention, the linker is a linear linker moiety (e.g., a succinyl moiety). In other embodiments, the linker is a cyclic or branched linker. Another example of a suitable linker for use with some conjugation methods is adipic acid dihydrazide (ADH).

Further, in embodiments of the invention, the drug is covalently linked to the N-terminus (e.g., the amino terminal group) and/or C-terminus (e.g., the carboxy group) of the flagellin adjuvant.

In the specific case of cocaine or a derivative thereof, these molecules can be conjugated to flagellin in a variety of ways. The following reaction conditions are for exemplary purposes only to illustrate the coupling of cocaine or cocaine derivatives to flagellin. Activated esters may be used and include without limitation 1-(3-dimethylaminopropyl)-3-ethyl-carbodimide (EDC), dicyclohexyl-carbodiimide (DCC) and 2,5,6-Cl₃(C₆H₂)COCl (Aldrich). Non-limiting examples of solvents include dimethylformamide (DMF), acetonitrile, methylene chloride, chloroform, ethylacetate and tetrahydrofuran. Various pH buffer systems can be used and temperatures and reaction times may vary, depending upon the drug-flagellin combination.

In one embodiment of the present invention, TA-CD is treated with 1-(3-dimethylaminopropyl)-3-ethyl-carbodimide (EDC) in the presence of N-hydroxysulpho-succinimide (sulpho-NHS) to create a semi-stable ester. The conjugate is formed by incubating this product with flagellin (see, e.g., Example 1).

In another embodiment, GND is treated with 1-(3-dimethylaminopropyl)-3-ethyl-carbodimide (EDC) in the presence of N-hydroxysulpho-succinimide (sulpho-NHS) to create a semi-stable ester. This product is then incubated with flagellin to create a conjugate (see, e.g., Example 2).

In yet another embodiment, conjugates of the present invention are synthesized by succinylating ecgonine methyl ester with succinic anhydride in DMF in the presence of one equivalent of triethylamine. The product is then coupled to the epsilon amino group of a lysine residue in flagellin using EDC (see, e.g., Example 3).

In still another embodiment, conjugates of the present invention are synthesized by reacting norcocaine with succinic anhydride in DMF in the presence of two equivalents of triethylamine. The product is then coupled to the epsilon amino group of a lysine residue in flagellin using EDC (see, e.g., Example 4).

Alternatively, succinylated norcocaine can be pre-activated by reacting it with 4-hydroxy-3-nitrobenzene sulfonic acid sodium salt, then conjugated to the epsilon amino group of a lysine residue in flagellin using EDC (see, e.g., Example 5).

Similarly, nicotine or its derivative can be modified and conjugated to flagellin in a variety of ways. The following are but a few exemplary procedures.

In one embodiment of the present invention, nicotine is conjugated with flagellin by utilizing the Mannich reaction (see, e.g., Example 6).

In yet another embodiment, nornicotine is succinylated prior to its conjugation to flagellin (see, e.g., Example 7).

Further, as another aspect, the invention provides compositions comprising two or more (e.g., two, three, four, five or more) conjugates of the invention. In embodiments of the invention, the composition comprises a combination of conjugates, either directed to the same drug (cross-immunization) or to multiple drugs (co-immunization, e.g., against cocaine and methamphetamine). Such mixtures of conjugates directed against multiple drugs are particularly useful in the prevention or treatment of polydrug abuse.

In representative embodiments, the composition does not comprise a virus particle or virus like particle (VLP). In embodiments of the invention, the composition does not comprise another protein carrier conjugated to an antigen (e.g., a drug of abuse or immunologically similar derivative thereof).

### V. Methods of Administration and Subjects

The present invention can be practiced for prophylactic and/or therapeutic purposes, in accordance with known techniques.

Without wishing to be bound by any particular theory of the invention, it appears that when a conjugate of the invention is administered to a subject (e.g., an addicted subject or a subject at risk of developing drug addiction), anti-drug antibodies specific to the drug are elicited. A therapeutic immunization regimen elicits and maintains sufficiently high titers of anti-drug antibodies, such that upon subsequent exposure to the drug, a neutralizing antibody response results in antibodies binding to a sufficient amount of the drug in order to reduce, if not eliminate, the pharmacological effects of the drug. For example, when the therapeutic composition is a cocaine derivative-flagellin conjugate, treatment induces an antibody response that is capable of reducing or neutralizing cocaine in the bloodstream and/or mucosal tissue of a subject, thereby reducing the addictive properties of the drug. Thus, delayed and/or reduced levels of the drug of abuse reach the central nervous system, and the subject receives diminished or no gratification from the use of the drug.

Further, in general, it appears there will be no significant adverse side effects due to immune complex formation. Specifically, the drug of abuse is generally small and monovalent and so is not able to cross-link antibody. Therefore, formation of immune complexes and the associated pathologies are not expected to occur after exposure to the drug of abuse.

Further, in embodiments of the invention, effective neutralization (protection) is long lasting and can be maintained for long periods of time, for example, for at least two, three, four, five six, nine or 12 months. This long-term effect substantially reduces or avoids compliance issues and resulting recidivism, which are problems with current therapies.

The invention can be practiced to produce an immune response against a drug of abuse in a subject, optionally a neutralizing immune response. Further, the invention can be practiced to prevent addiction to a drug of abuse in a subject (e.g., a subject a risk for developing addiction) or to treat addiction in a subject addicted to a drug of abuse.

Subjects to be treated by the methods of the invention can include both avian and mammalian subjects, mammalian subjects including but not limited to humans, non-human mammals, non-human primates (e.g., monkeys, baboons, and chimpanzees), dogs, cats, goats, horses, pigs, cattle, sheep, and the like, and laboratory animals (e.g., rats, mice, rabbits, gerbils, hamsters, and the like). Avian subjects include without limitation chickens, geese, ducks, quail and pheasant and birds kept as domestic pets.

Suitable subjects include both males and females and subjects of all ages including infant, juvenile, adolescent, adult and geriatric subjects. Subjects may be treated for any purpose, such as for eliciting an immune response; for eliciting the production of antibodies in that subject, which antibodies can be collected and used for other purposes such as research or diagnostic purposes or for administering to other subjects to produce passive immunity therein and/or to prevent or treat addiction to a drug of abuse, etc.

In particular embodiments, the subject is “in need of the methods of the invention, e.g., because the subject is addicted to a drug of abuse or is at risk of becoming addicted to a drug of abuse.

Accordingly, in particular embodiments, the invention provides a method of producing an immune response (optionally, a neutralizing immune response) against a drug of abuse in a subject (e.g., a mammalian subject), the method comprising administering a conjugate, composition or immunogenic formulation of the invention to the subject in an amount effective to produce an immune response in the subject against the drug of abuse. In embodiments, the invention is practiced to produce an immune response against two or more (e.g., two, three, four, etc.) drugs of abuse.

As another aspect, the invention provides a method of preventing addiction to a drug of abuse in a subject (e.g., a mammalian subject), the method comprising administering a conjugate, composition or immunogenic formulation of the invention to the subject in an amount effective to prevent addiction to a drug of abuse in the subject. In embodiments, the invention is practiced to prevent addiction to two or more (e.g., two, three, four, etc.) drugs of abuse.

The invention further provides a method of reducing the effect of a drug of abuse in a subject (e.g., a mammalian subject), the method comprising administering a conjugate, composition or immunogenic formulation of the invention to the mammalian subject in an amount effective to reduce the effect of the drug of abuse in the mammalian subject upon subsequent challenge with the drug of abuse. In embodiments of the invention, the effect is a physiological effect, psychological effect and/or a behavioral effect. For example, in embodiments of the invention, the methods and immunogenic formulations of the invention result in the production of a sufficiently high titer of neutralizing antibodies to the drug such that upon subsequent challenge with the drug the antibodies are capable of reducing the effects and/or addictive nature of the drug in the subject. In embodiments, the invention is practiced to reduce the effects of two or more (e.g., two, three, four, etc.) drugs of abuse.

Also provided is a method of treating drug addiction in a subject (e.g., a mammalian subject), the method comprising administering a conjugate, composition or immunogenic formulation of the invention to a subject addicted to a drug of abuse in an amount effective to reduce the addiction in the subject to the drug of abuse. In embodiments, the invention is practiced to treat addiction to two or more (e.g., two, three, four, etc.) drugs of abuse.

The invention also contemplates a method of reducing the amount or concentration of a drug of abuse (and/or its active metabolite) in the central nervous system (e.g., brain) of a subject (e.g., a mammalian subject), the method comprising administering a conjugate, composition or immunogenic formulation of the invention to a subject in an amount effective to reduce the amount or concentration of the drug of abuse in the central nervous system of the subject. In embodiments, the invention is practiced to reduce the amount or concentration of two or more (e.g., two, three, four, etc.) drugs of abuse in the central nervous system (e.g., brain).

The methods of the invention generally result in activation of TLR5 on antigen presenting cells (e.g., dendritic cells). In addition, in embodiments of the invention, the methods of the invention induce cellular and/or humoral immune responses. In embodiments of the invention, the methods result in the induction of innate immunity. In embodiments of the invention, the methods activate naïve T cells and/or memory T cells. In embodiments of the invention, the methods result in interaction of antigen-presenting cells and flagellin-specific naïve and/or memory T cells. In embodiments of the invention, the methods result in interaction of activated flagellin-specific helper T cells and B cells specific to the drug of abuse. In embodiments of the invention, the methods result in the conjugate being processed via the Class II MHC antigen-processing pathway.

In representative embodiments, the invention is practiced to induce an immune response against cocaine. In embodiments of the invention, the present invention is practiced to elicit anti-cocaine antibodies capable of binding free drug and preventing transit of the drug to the reward system in the brain thereby reducing addictive drug-taking behavior. It is believed that cocaine affects the neuronal uptake of dopamine, norepinephrine, and serotonin. While not intending to exclude other modes of action, it is believed that once cocaine enters the blood stream following inhalation (snorting or smoking) or intravenous administration, it rapidly crosses the blood-brain barrier where the intact cocaine binds to specific recognition sites located on the dopamine transporter of mesolimbocortical neurons, thereby inhibiting dopamine reuptake into presynaptic neurons. The euphoric rush is due to rapid build-up of dopamine in the synapse. For this reason, cocaine remains the most complex and challenging, and before the present invention, elusive drug for which therapy is sought. Although estimates vary, it is believed that following intranasal administration, changes in mood and feeling states are perceived within about 2 to 5 minutes, and peak effects occur at 10 to 20 minutes. Cocaine free-base, including the free-base prepared with sodium bicarbonate (crack), has a relatively high potency and rapid onset of action, approximately 8 to 10 seconds following smoking. Due to the route of the circulation, intravenous cocaine is intermediate in time of onset of euphoria taking from about 30 seconds to about 1 minute.

Thus, in embodiments of the invention, the conjugates, immunogenic formulations and methods of the invention result in the production of antibodies that can protect the subject (partially or completely) from the effects of a subsequent cocaine challenge (in other words can partially or completely neutralize the effects of cocaine) within these time frames, e.g., within about 4, 5, 6, 7, 8, 9, 10, 12, 15, 18, 20, 30, 45 seconds or 1, 2, 3, 4, 5, 6, 7, 8, 9, 10, 12, 15, 18, 20, 25, 30, 40, 50 or 60 minutes of administration of the drug challenge.

In embodiments of the invention, a neutralizing anti-drug antibody response is elicited and maintained such that the effect on the subject by a subsequent challenge with the drug is reduced (partially or completely eliminated). Optionally, the neutralizing anti-drug antibody response is maintained in the subject for at least about two, three, four, five, six, nine or even 12 months or longer after the last administration of the conjugates, compositions and immunogenic formulations of the invention. Suitable ranges include from about two months to six months, about nine months to twelve months, from about three months to six, nine months or twelve months, from about four months to six, nine or twelve months, and from about six months to nine months or twelve months.

Moreover, in embodiments of the invention, high titers of antigen-specific IgG (e.g., in blood, plasma or serum) are achieved, e.g., above about 1×10⁴, 1×10⁵ or even 2.5×10⁵ or higher.

Immunogenic formulations for use in the inventive methods are described below. Boosting dosages can further be administered over a time course of days, weeks, months or years.

Any suitable dosage of the conjugates of the invention can be administered to give the desired response. In particular embodiments, the dosage of the conjugates of the present invention ranges from at least about 0.01, 0.05, 0.1, 0.5, 1, 5, 10, 15, 20, 25, 30, 50, 75, 100, 150, 200 or 250 μg to about 5, 10, 15, 20, 25, 30, 50, 75, 100, 150, 200, 250, 300, 500 or 1000 μg for a typical (e.g., 70 kg) subject (including any combination of the lower and upper dosages as long as the lower value is less than the upper value).

An exemplary dosage scheme is administration about once every week, once every two weeks, once every 3 weeks, once a month, once every 2 months, once every 3 months, once every 4 months, once every 6 months, once every 9 months, once a year, or once every two years or even longer intervals in between administrations.

As another illustration, a primary immunization can be given followed by a second boosting immunization at around 2, 4, 6 or 8 weeks, followed by further boosting immunizations every two, three, four, or six months thereafter.

In embodiments of the invention, administration is no more often than about once a month, no more often than about once every two months, no more often than about once every three months, no more often than about once every six months (e.g., twice a year), no more often than about once a year, no more often than about once every two years, or even longer intervals between administrations.

In embodiments of the invention, a single dosage is administered. In embodiments of the invention, two or more (e.g., two, three, four, five or six) dosages are administered.

In representative embodiments, about two, three, four, five or six dosages are administered the first year, and about one, two, three or four dosages yearly are administered thereafter.

Periodic tests of plasma from vaccinated subjects may be useful to determine individual effective doses and dosing frequency. Titer levels are increased and maintained through periodic boosting.

It is specifically intended that the invention encompass any combination of the number of dosages, dosing frequency and dosage amounts described herein.

The methods of the invention can also comprise administering other immunogenic agents (e.g., directed against other antigens, such as cancer antigens or antigens from a pathogenic organism or virus) or agents useful in the prevention or treatment of drug addiction. The additional agent(s) can be administered in the same composition as the flagellin conjugate of the invention. Alternatively, the additional agent can be administered separately, concurrently or serially. As used herein, the term “concurrent” or “concurrently” means sufficiently close in time to produce a combined effect (that is, simultaneously or two or more events occurring within a short time period before or after each other).

In embodiments of the invention, two or more (e.g., three, four or five) conjugates of the invention can be administered, directed to the same drug or to multiple drugs. The two or more conjugates can be delivered in the same and/or separate compositions, concurrently and/or serially.

Administration of the conjugates, compositions and immunogenic formulations of the invention can be by any route known in the art. As non-limiting examples, the route of administration can be by inhalation (e.g., oral and/or nasal inhalation), oral, buccal (e.g., sublingual), rectal, vaginal, topical (including administration to the airways), intraocular, transdermal, by parenteral (e.g., intramuscular [e.g., administration to skeletal muscle], intravenous, intra-arterial, intraperitoneal and the like), subcutaneous, intradermal, intrapleural, intracerebral, and/or intrathecal routes.

In particular embodiments, administration of the conjugates, compositions and immunogenic formulations of the invention is to a mucosal surface, e.g., by intranasal, inhalation, intra-tracheal, oral, buccal (e.g., sublingual), intra-ocular, rectal or vaginal administration, and the like. In general, mucosal administration refers to delivery to a mucosal surface such as a surface of the respiratory tract, gastrointestinal tract, urinary tract, reproductive tract, etc.

Methods of administration to the respiratory tract include but are not limited to transmucosal, intranasal, inhalation, bronchoscopic administration, or intratracheal administration or administration to the lungs.

The conjugates, compositions, and immunogenic formulations of the invention can be administered to the lungs of a subject by any suitable means, optionally by administering an aerosol suspension of respirable particles comprising the fusion protein, composition, or immunogenic formulation which the subject inhales. The respirable particles can be liquid or solid. Aerosols of liquid particles comprising a fusion protein, composition, or immunogenic formulation of the invention may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the can likewise be produced with any solid particulate medicament aerosol generator, by techniques known in the pharmaceutical art.

Immunomodulatory compounds, such as immunomodulatory cytokines and chemokines (for example, as described above), such as CTL inductive cytokines, can be administered concurrently to a subject.

Cytokines may be administered by any method known in the art. Exogenous cytokines may be administered to the subject, or alternatively, a nucleic acid encoding a cytokine may be delivered to the subject using a suitable vector, and the cytokine produced in vivo. In particular embodiments, the cytokine is provided as a part of a fusion protein of the invention. For example, a fusion protein comprising a flagellin adjuvant and an immunomodulatory cytokine (e.g., interferon-γ) can be administered (or a nucleic acid encoding the same). Optionally, the fusion protein is conjugated to a drug of addiction or immunologically similar derivative.

In addition to their use for prophylactic or therapeutic purposes, the conjugates, compositions, and immunogenic formulations of the present invention can be administered to subjects for the purpose of producing antibodies to a drug of addiction, which antibodies are in turn useful for research, diagnostic or to provide passive immunity to a subject.

Thus, the invention also contemplates passive immunization methods, which encompass administration of or exposure to an intact anti-drug antibody or polyclonal antibody or monoclonal antibody fragment (such as Fab, Fv, (Fab′)₂ or Fab′) prepared using the conjugates, compositions or immunogenic formulations of the instant invention. Passive immunization can be particularly useful as an initial co-treatment and/or a supplementary complementary treatment (for example, during the period of time after initial administration of the vaccine but before the body”s own production of antibodies) or in acute situations to prevent death (for example, when a person presents with a drug overdose). In some situations, passive therapy alone may be used, such as when the patient is immunocompromised or needs a rapid treatment.

The invention also encompasses the use of a conjugate, composition or immunogenic formulation of the invention in the manufacture of a medicament to prevent or treat drug addiction in a mammalian subject.

Also provided is a conjugate, composition or immunogenic formulation of the invention to prevent or treat drug addiction in a mammalian subject.

The methods of the invention can be used in conjunction with counseling, conventional pharmacological approaches and/or passive immunization. Further, the methods can be practiced to immunize against a single drug or several drugs simultaneously or in succession.

### VI. Pharmaceutical Formulations

The invention further provides pharmaceutical formulations (e.g., immunogenic formulations) comprising a conjugate or composition of the invention in a pharmaceutically acceptable carrier. In particular embodiments, the pharmaceutical formulation is formulated for mucosal, intradermal, intramuscular or subcutaneous delivery. By “pharmaceutically acceptable” it is meant a material that is not toxic or otherwise undesirable.

In representative embodiments, the conjugate or composition is present in the pharmaceutical formulation in an “immunogenically effective” amount. An “immunogenically effective amount” is an amount that is sufficient to evoke an active immune response (i.e., cellular and/or humoral) in the subject to which the pharmaceutical formulation is administered, optionally a neutralizing immune response (e.g., to prevent and/or treat addiction). The degree of protection or neutralization conferred need not be complete or permanent, as long as the benefits of administering the pharmaceutical formulation outweigh any disadvantages thereof. Immunogenically effective amounts depend on the conjugate, the particular drug of abuse, the manner of administration, the severity of the addiction being treated, and the judgment of the prescribing physician and other factors known by those skilled in the art.

Unless indicated otherwise, the flagellin adjuvants of the invention are administered per se and not as part of live, killed, or recombinant bacterium- or virus-vectored vaccine. Further, unless indicated otherwise, the flagellin adjuvants of the invention are isolated flagellins and flagellin fusion proteins, e.g., are not incorporated into flagella.

Likewise, unless indicated otherwise, the conjugates of the invention are administered per se and not as part of live, killed, or recombinant bacterium- or virus-vectored vaccine. Further, unless indicated otherwise, the conjugates of the invention are not incorporated into flagella.

Dosages of pharmaceutically active substance can be determined by methods known in the art, see, e.g., Remington's Pharmaceutical Sciences (Maack Publishing Co., Easton, Pa.). In particular embodiments, the dosage of the conjugates of the present invention ranges from at least about 0.1, 0.5, 1, 5, 10, 15, 20, 25, 30, 50, 75, 100, 150, 200 or 250 μg to about 5, 10, 15, 20, 25, 30, 50, 75, 100, 150, 200, 250, 300, 500 or 1000 μg for a typical (e.g., 70 kg) subject (including any combination of the lower and upper dosages as long as the lower value is less than the upper value). The initial dose can be followed by one or more boosting dosages over weeks, months or years.

Optionally, the conjugate is present in an immunogenically effective amount, as defined herein. Further, in some embodiments, the flagellin adjuvant is present in an “adjuvant effective amount.” An “adjuvant effective amount” is an amount of the flagellin adjuvant that is sufficient to enhance or stimulate the active immune response (cellular and/or humoral) mounted by the host against the drug of abuse or derivative thereof. In particular embodiments, the active immune response (e.g., humoral and/or cellular immune response) by the host is enhanced by at least about 2, 3, 4, 5, 10, 15, 20, 30, 40, 50, 60, 75, 100, 150, 500, 1000-fold or more. In other embodiments, an “adjuvant effective amount” is an amount of the flagellin adjuvant that reduces the amount of antigen required to achieve a specified level of immunity (cellular and/or humoral), optionally mucosal immunity, for example, a reduction of at least about 15%, 25%, 35%, 50%, 65%, 75%, 80%, 85%, 90%, 95%, 98% or more in the amount of antigen. As a further option, an “adjuvant effective amount” can refer to an amount of the flagellin adjuvant that accelerates the induction of the immune response in the host and/or reduces the need for booster immunizations to achieve protection or neutralization. As yet another alternative, an “adjuvant effective amount” can be an amount that prolongs the time period over which an immune response, optionally a neutralizing immune response, is sustained (e.g., by at least about a 2-fold, 3-fold, 5-fold, 10-fold, 20-fold longer time period or more).

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

While adjuvants beyond flagellin are generally not required, the composition can optionally comprise an additional adjuvant, such as complete or incomplete Freund's adjuvant, aluminum phosphate, aluminum hydroxide, alum, cytokines, TLR ligands, and the like. In embodiments of the invention, the adjuvant in the composition consists essentially of or consists of the flagellin adjuvant. In representative embodiments, the composition does not comprise an adjuvant other than the flagellin adjuvant.

The concentration of the conjugate in the pharmaceutical formulations can vary widely, e.g., from less than about 0.01% or 0.1% up to at least about 2% to as much as 20% to 50% or more by weight, and will be selected primarily by fluid volumes, viscosities, etc., in accordance with the particular mode of administration selected.

The conjugate can be formulated for administration in a pharmaceutical carrier in accordance with known techniques. See, e.g., Remington, The Science And Practice of Pharmacy (9^{th} Ed. 1995). In the manufacture of a pharmaceutical composition according to the invention, the conjugate(s) is typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. One or more conjugates can be incorporated in the formulations of the invention, which can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

In particular embodiments, the pharmaceutical formulation is administered to a mucosal surface, e.g., by intranasal, inhalation, intratracheal, oral, buccal, rectal, vaginal or intra-ocular administration, and the like.

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, for example, 1-10% by weight. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, for example, 0.25-5% by weight. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules containing the protein(s), optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles comprising the compound(s) in an inert base such as gelatin and glycerin or sucrose and acacia.

Pharmaceutical formulations suitable for parenteral administration can comprise sterile aqueous and non-aqueous injection solutions of the proteins, which preparations are generally isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration can be prepared as unit dose suppositories. These can be prepared by admixing the protein(s) with one or more conventional solid carriers, such as for example, cocoa butter and then shaping the resulting mixture.

In embodiments of the invention, pharmaceutical formulations suitable for topical application to the skin can take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of an optionally buffered aqueous solution of the compound(s). Suitable formulations can comprise citrate or bis\tris buffer (pH 6) or ethanol/water and can contain from 0.1 to 0.2M active ingredient.

Further, the conjugate can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

The present invention is more particularly described in the following examples that are intended as illustrative only since numerous modifications and variations therein will be apparent to those skilled in the art.

### Example 1

### Conjugation of TA-CD to Flagellin

TA-CD was synthesized and then conjugated to flagellin in a two-step process. The TA-CD is activated in dimethylformamide (DMF) (200 μl) using an aqueous solution of 1-(3-dimethylaminopropyl)-3-ethyl-carbodimide (EDC) (26 μmol) and N-hydroxysulfo-succinimide (sulfo-NHS) (26 μmol). The aqueous content is 10%. After 20 hours at 22° C., flagellin (about 1 to 5 mg in 4 ml of 50 nM phosphate buffer (PB), pH 7.5) is added, and the solution is kept at 4° C. for 20 hours. The conjugate is dialyzed against two changes of 100 mM PB, pH 7.0. The resultant product contained approximately 5 molecules of TA-CD per molecule of flagellin. In addition, we also conjugated TA-CD to bovine serum albumin (BSA) and ovalbumin (OVA).

### Example 2

### Conjugation of GND to Flagellin

GND is synthesized using the method of Sakurai et al., Tetrahedron Lett. 37: 5479-5482 (1996). The GND is activated in dimethylformamide (DMF) (200 μl) using an aqueous solution of 1-(3-dimethylaminopropyl)-3-ethyl-carbodimide (EDC) (26 μmol) and N-hydroxysulfo-succinimide (sulfo-NHS) (26 μmol). The aqueous content is 10%. After 20 hours at 22° C., flagellin (about 1 to 5 mg in 4 ml of 50 nM phosphate buffer (PB), pH 7.5) is added, and the solution is kept at 4° C. for 20 hours. The conjugate is dialyzed against two changes of 100 mM PB, pH 7.0.

### Example 3

### Conjugation of Succinylated Ecgonine Methyl Ester to Flagellin

Triethylamine (0.16 mmol), followed by succinic anhydride (0.16 mmol), is added to a solution of ecgonine methyl ester (0.16 mmol) in DMF (2 ml). The solution is heated at 35° C. for 2 hours. Solvent is removed under reduced pressure, and the residue is purified by silica gel flash chromatography (9:1 chloroform:methanol as the eluent). 2.4 mg of the resultant hemisuccinate, a white powder, is solubilized in 0.5 ml distilled water and chilled to 0° C. EDC (7.69 μmol) is added to the chilled hemisuccinate solution. After 10 minutes, flagellin (about 1 to 5 mg in 0.5 ml PBS) is added, and the solution is allowed to warm to ambient temperature overnight. The conjugate is dialyzed against two changes of 100 mM PB, pH 7.0.

### Example 4

### Conjugation of Succinylated Norcocaine to Flagellin

Triethylamine (6.14 mmol), followed by succinic anhydride (6.14 mmol), is added to a solution of norcocaine hydrochloride (3.07 mmol) in DMF (20 ml). The solution is heated at 45° C. overnight. Solvent is removed under pressure, and the residue is purified by silica gel flash chromatography (2:1 chloroform:methanol as the eluent). 14 mg of the resultant acid, a thick syrup, is solubilized in 1 ml distilled water and chilled to 0° C. EDC (0.055 mmol) is added to the chilled acid solution. After 5 minutes, flagellin (about 1 to 5 mg in 1.0 ml PBS) is added, and the solution is allowed to warm to ambient temperature overnight. The conjugate is dialyzed against two changes of 100 mM PB, pH 7.0.

### Example 5

### Conjugation of Pre-Activated, Succinylated Norcocaine to Flagellin

Dicyclohexylcarbodimide (DCC) (0.31 mmol) is added to succinylated norcocaine (0.26 mmol) in DMF (5 ml). After 10 minutes, 4-hydroxy-3-nitrobenzene sulfonic acid sodium salt (0.31 mmol) is added. The resulting yellow solution is kept at ambient temperature for 4 days, at which point it is filtered under reduced pressure. The filtrate is added to cold diethyl ether (10 ml) with vigorous stirring. Hexane (5 ml) is added, and after complete precipitation of a yellow oil, the colorless supernatant is decanted off. This process is repeated, and the oil is dried overnight under reduced pressure. The resultant ester is dissolved in DMF (100 μl) and added dropwise to flagellin (about 1 to 5 mg in 2 ml PB) at 4° C., then warmed to ambient temperature. The conjugate is dialyzed against two changes of 100 mM PB, pH 7.0.

### Example 6

### Conjugation of Nicotine to Flagellin-Formaldehyde

Flagellin (about 1 to 5 mg) and nicotine-HCl (approximately 1 mg) are added to 400 μl 2-(N-morpholino)ethanesulfonic acid (MES) buffer, pH 4.5, 0.15 M NaCl. 50 μl of 37% formaldehyde is added, and the solution is allowed react at 37° C. for 3 hours. The solution is dialyzed against water overnight.

### Example 7

### Conjugation of Succinylated Nornicotine to Flagellin

Triethylamine (75 mmol), followed by succinic anhydride (100 mmol), is added to a solution of nornicotine (50 mmol in methylene chloride). The solution is heated at reflux for 18 hours. The reaction mixture is washed sequentially with 10% aqueous hydrochloric acid, saturated sodium bicarbonate solution, brine and water. Solvent is removed under reduced pressure, and the residue is purified by silica gel flash chromatography (with chloroform:methanol as the eluent). The resultant succinylated nornicotine (5 μmol) is solubilized in 0.1 ml DMF. Diisopropylethylamine (10 μmol) is added to the solution, followed by O-(7-Azabenzotriazol-1-yl)-1,1,3,3-tetramethyluronium hexafluorophosphate (HATU) (5.5 μmol). After 10 minutes, the pale yellow solution is added dropwise to flagellin (about 1 to 5 mg in 0.9 ml of 0.1 M sodium borate buffer, pH 8.8), and the mixture is stored at ambient temperature for 18 hours. The pH of the conjugate solution is adjusted to pH 7.0 by careful addition of 0.1 M aqueous hydrochloric acid. The conjugate is dialyzed against two changes of 100 mM PB, pH 7.0, then filtered through a 0.2 μm filter.

### Example 8

### Stability of Flagellin-Hapten Conjugates

To assess the stability of flagellin-hapten conjugates, purified samples of the conjugate are incubated at −80° C., −20° C., or 4° C. for 1,4,12,24, 36, and 52 weeks. Aliquots are assayed for 1) protein content, 2) size retention as measured via SDS PAGE, 3) in vitro TLR5-stimulating activity (as described in Mizel et al., Clin. Vaccine Immunol. 16:21-28 (2009)), and 4) in vivo immunogenicity in BALB/c mice (2 i.m. immunizations followed by assessment of circulating anti-cocaine IgG).

### Example 9

### Retention of Flagellin Signaling via TLR5

To assess the ability of the TA-CD-flagellin conjugate to signal via TLR5, the TA-CD-flagellin conjugate made as described in Example 1 was used to stimulate RAW 424 cells, a cell line that has been stably transfected with a murine TLR5 cDNA, as well as RAW 264.7 cells, the parent cell line used to create the RAW 424 cell line. If TLR5 signaling is retained in the conjugate, then stimulated RAW 424, but not RAW 424.7 cells, should produce high levels of the cytokine TNF alpha. We found that the TA-CD-flagellin retained full TLR5 signaling activity.

### Example 10

### Immunization Studies with TA-CD-Flagellin

Having established our ability to generate a TA-CD-flagellin conjugate that retained TLR5 signaling activity, we next initiated immunization studies in mice and rats. TA-CD-flagellin was made as described in Example 1. TA-CD-BSA was used as a control (Example 1). Thus, groups of 7 mice and rats received 1 microgram of TA-CD-BSA or TA-CD-flagellin intra-muscularly on days 0 and 28 and were bled on day 42. Plasma was prepared and assessed for anti-TA-CD IgG using ELISA plates coated with TA-CD-OVA. Four of the mice given TA-CD-flagellin had anti-TA-CD IgG titers above 1×10⁶, whereas three others had titers of 3×10⁶, 5×10⁵ and 1×10⁶. None of the mice given TA-CD-BSA had titers above 1×10³ (the highest dilution of plasma tested). Rats given TA-CD-flagellin had titers ranging from 5×10⁴ to 1×10⁶, with most of the animals well above 1×10⁶. Only one rat given TA-CD-BSA had a measurable titer (3×10⁴). This rat had some trauma at the site of injection and thus the higher titer in this single control animal might be due to activation of nonspecific innate immunity. The titers achieved with TA-CD-flagellin in the mice and rats are considerably higher than have been reported for other experimental cocaine vaccines.

Based on preliminary data, we believe that the affinity of the antibodies for cocaine are not yet of sufficient magnitude to be effective in neutralizing cocaine in the bloodstream.

Nonetheless, our initial results clearly establish that flagellin is a highly effective and potent adjuvant and carrier for a cocaine analog vaccine. The ability to retain full TLR5 signaling activity (Example 9) after conjugation to TA-CD as well as the very high titers of induced IgG represent substantial advances over currently available approaches. Additional immunizations (e.g., two, three or four) may achieve antibodies of very high affinity and titer that will provide substantial neutralizing activity against cocaine.

### Example 11

### Immunization Studies with GND-Flagellin

The cocaine analog GND should be able to generate high titer antibodies with extremely high affinity for cocaine and, thus, should be highly effective in neutralizing cocaine bioactivity and access to the brain. BALB/c mice are immunized intramuscularly on days 0 and 28 with GND-flagellin conjugate and then evaluated 14, 28, 56, and 168 days later for circulating levels of anti-cocaine IgG using a standard ELISA. These experiments are used to establish the minimal dose of vaccine required to elicit the maximal anti-cocaine IgG response as well as the stability of the induced antibody titers and the affinity for cocaine.

### Example 12

### Toxicology Studies

Groups of 20 female and male BALB/c mice are immunized i.m. with the hapten-flagellin conjugate (e.g., a cocaine derivative such as TA-CD or GND), flagellin, or PBS. During the first 24 hr after the primary immunization, all mice are assessed for behavioral changes (for example, difficulty in breathing, matted fur, hunched posture, and lack of mobility). A subset of the mice in each group are sacrificed at 24 hr and evaluated for circulating levels of TNFα (a measure of systemic inflammation) and histology at the site of injection (a measure of the local inflammatory response). Liver enzymes, kidney function, glucose, phosphorus, calcium, and total protein are analyzed, along with a complete blood count. These analyses are repeated after a boost immunization on Day 28 and at 60 days after the boost.

### Example 13

### Cocaine-Induced Locomotor Activity in Mice Immunized with a Cocaine Analog-Flagellin Conjugate

Mice are immunized on days 0 and 28 with either a cocaine derivative-flagellin (e.g., TA-CD or GND) conjugates or just flagellin (control group). After assessing the circulating titers of anti-cocaine IgG in the control and immunized animals, various doses of cocaine are administered (appropriate doses will vary, depending on the strain and species). Mice are tested in locomotor activity chambers.

Following behavioral testing, the animals are euthanized (5, 10, 30, or 60 minutes later). Trunk blood is collected from each animal, and the forebrain is removed. Tissue and plasma samples are extracted with organic solvents and isolated by reverse phase HPLC. Levels of cocaine and its metabolites (including norcocaine, benzoylecognine and norbenzyoylecognine) are assessed by absorbance at 235 nm.

Alternatively, plasma and brain levels of cocaine are determined by measuring the amount of radioactivity present in the plasma and brains of mice injected with [3H]cocaine.

### Example 14

### Cocaine Self-Administration in Rats Immunized with a Cocaine Analog-Flagellin Conjugate

The following are exemplary conditions for characterizing the effects of a cocaine derivative-flagellin conjugate of the present invention.

### A. Fixed Ratio Schedule

Rats are trained to self-administer cocaine on a fixed ratio schedule in which a cocaine injection is presented after each response on a lever. Rats are implanted with chronically indwelling jugular catheters and given access to cocaine on an fixed ratio schedule during daily 2 h sessions. The animals “load up” on cocaine at the beginning of the 2 h session and then continue to respond at a precise rate which maintains a constant (preferred) blood level.

Following acquisition of stable rates of intake at a training dose of 0.75 mg/kg/inj, subjects are tested with various doses of cocaine (20, 30, 60, 110, 200, 350, and 630 μg/kg/inj). After the dose-effect curve has been established, animals are randomly assigned to control or vaccine groups. Animals in the vaccine group receive two intramuscular immunizations with the cocaine derivative-flagellin conjugate (day 0 and 28). After an additional two weeks, the seven point dose-effect curve is re-assessed each week for 3 weeks.

### B. Progressive Ratio Schedule

Under a progressive ratio schedule, the first drug injection of the daily session “costs” only a single response, but this response requirement increases with each successive injection through a log series (i.e. 1, 2, 4, 6, 9, 12, 15, 20, 25, 32, 40, 50, 62, 77, 95, 118, 145, 178, 219, 268, 328, 402, 492, 603 . . . ) until the animal ceases to respond. The point at which the animal will no longer pay for the next injection is called the ‘break point”.

Rats are trained on a progressive ratio schedule and a dose-effect curve generated (0.19, 0.38, 0.75, 1.5 mg/kg/inj). Subjects are then randomly assigned to the control or vaccine group. The dose-effect curve is re-evaluated 2, 3 and 4 weeks after vaccination.

### C. Cocaine-Induced Relapse

In this model, inhibition of cocaine-induced relapse is investigated using standard procedures. Rats previously trained to self-administer cocaine undergo an extinction procedure. That is, responding no longer produces a cocaine injection with the result that animals gradually stop pressing on the lever. Interestingly, it has been repeatedly shown that an IP injection of cocaine (15 mg/kg) reinstates an animal's interest in the lever and this increase in responding can be used as an index of ‘relapse’ or ‘drug seeking’.

Here, cocaine-induced relapse at 2, 4 and 6 weeks is evaluated according to well established behavioral protocols (see Carerra et al., Proc. Natl. Acad. Sci. 97:6202-6206 (2000)) after two immunizations in control and cocaine analog-flagellin vaccinated groups.

### Example 15

### Extracellular Dopamine Concentrations in Nonhuman Primates Immunized with a Cocaine Analog-Flagellin Conjugate

Cocaine exerts its actions by binding to dopamine, serotonin and norepinephrine transporters and blocking reuptake of these neurotransmitters, thereby increasing their levels in the brain. A number of studies have demonstrated a strong relationship between the reinforcing effects of cocaine and its ability to increase concentrations of extracellular dopamine. PET imaging studies using tracer doses of tracers such as [¹¹C]raclopride and [¹⁸F]fluoroclebopride (FCP) in humans and monkeys (see, for example, Mach et al., Pharmacol. Biochem. Behav. 57:477-486 (1997)) have shown that elevations in dopamine produced by drugs such as cocaine produce dramatic reductions in D₂ binding potentials (decreases in receptor availability resulting from the increased occupation by dopamine). The following protocol seeks to take advantage of this property by comparing the effects of an iv injection of cocaine before and after immunization with the cocaine derivative-flagellin conjugate.

Cynomolgus monkeys are tested at three distinct time points: 1) prior to cocaine challenge (control scan), 2) following cocaine challenge, prior to immunization, and 3) after immunization and second cocaine challenge. Each testing period consists of a bolus injection of the D₂ radiotracer [¹⁸9 FOP, followed by PET scans acquired over the subsequent 3-hour period. For cocaine challenges, radiotracer injections are followed by i.v. injections of 1.0 mg/kg cocaine (previously shown to reduce uptake by as much as 40% (Mach et al., Pharmacol. Biochem. Behav. 57:477-486 (1997))) at a time point when FCP uptake is maximal, thereby providing a stable baseline for examining the effects of cocaine on radiotracer washout. Monkeys are immunized with either a cocaine derivative-flagellin conjugate or flagellin only control.

### Example 16

### Assay of Cocaine Levels in Rodent Tissues

The assay of cocaine and metabolites in rat tissues provide a chemical determination of whether cocaine enters the brain following production of anti-cocaine antibodies. Optimal separation of cocaine and metabolites was obtained using high pressure liquid chromatography and provides an accurate determination of cocaine and benzoylecognine down to 10 ng, a sensitivity that is far higher than that needed to analyze these compounds in animal tissues following injection of cocaine.

### Example 17

### Development of a GMP Compatible Purification Protocol for Flagellin

As a first step in the GMP production of a cocaine analog-flagellin conjugate, we developed a simple purification process for flagellin. BL21 (DE3) bacteria carrying the inducible flagellin plasmid are induced with IPTG for 3 hours when the bacteria have reached stationary phase of growth. This promotes retention of flagellin in inclusion bodies. The inclusion bodies are then purified by centrifugation. The flagellin is extracted using 5M guanidine HCl, dialyzed into a low ionic strength buffer and then subjected to CM-Sepharose fast flow ion exchange chromatography. The peak fractions containing the flagellin are pooled, and the material concentrated before passage through an Acrodisc® membrane that removes endotoxin and nucleic acids. The purity of the product is greater than 90%.

The foregoing is illustrative of the present invention, and is not to be construed as limiting thereof. The invention is defined by the following claims, with equivalents of the claims to be included therein.

1. A conjugate comprising a flagellin adjuvant covalently linked to a drug of abuse or an immunologically similar derivative thereof.

2. The conjugate of claim 1, wherein the drug of abuse or immunologically similar derivative thereof is cocaine, a barbiturate, a benzodiazepine, flunitrazepam, gamma-hydroxybutyrate, methaqualone, a ketamine, phencyclidine, lysergic acid diethylamide, mescaline, psilocybin, heroine, morphine, opium, thebaine, hydromorphone, oxycodone, hydrocodone, fentanyl, codeine, an amphetamine, methylenedioxy-methamphetamine, methamphetamine, methcathinone, methylphenidate, meperidine, pentazocine, delta-9-tetrahydrocannabinol (THC), nicotine and/or an immunologically similar derivative of any of the foregoing.

3. The conjugate of claim 1, wherein the drug of abuse or derivative thereof is cocaine or a cocaine derivative.

4. The conjugate of claim 3, wherein the cocaine derivative is TA-CD.

5. The conjugate of claim 3, wherein the cocaine derivative is GND.

6. The conjugate of claim 1, wherein the flagellin adjuvant comprises:
(a) a flagellin N-terminal constant region; and
(b) a flagellin C-terminal constant region.

7. The conjugate of claim 1, wherein the flagellin adjuvant comprises a deleted flagellin hypervariable region or the flagellin hypervariable region is absent from the flagellin adjuvant.

8. A method of making the conjugate of claim 1, wherein the method comprises covalently linking a drug of abuse or immunologically similar derivative thereof to a flagellin adjuvant.

9. An immunogenic formulation comprising the conjugate of claim 1 in a pharmaceutically acceptable carrier.

10. An article of manufacture comprising a closed, pathogen-impermeable container and a sterile preparation enclosed within said container, wherein said preparation comprises the immunogenic formulation of claim 9.

11. A method of producing an immune response against a drug of abuse in a mammalian subject, the method comprising administering the conjugate of claim 1 to the mammalian subject in an amount effective to produce an immune response in the mammalian subject against the drug of abuse.

12. A method of reducing the effect of a drug of abuse in a mammalian subject, the method comprising administering the conjugate of claim 1 to the mammalian subject in an amount effective to reduce the effect of the drug of abuse in the mammalian subject.

13. A method of reducing the amount of a drug of abuse in the brain of a mammalian subject, the method comprising administering the conjugate of claim 1 to the mammalian subject in an amount effective to reduce the amount of the drug of abuse in the brain of the mammalian subject.

14. The method of claim 11, wherein the subject is addicted to the drug of abuse.

15. A method of treating drug addiction in a mammalian subject, the method comprising administering the conjugate of claim 1 to a mammalian subject addicted to a drug of abuse in an amount effective to reduce the addiction in the mammalian subject to the drug of abuse.

16. The method of claim 11, wherein the administering step is carried out by intramuscular delivery, intradermal delivery or subcutaneous delivery.

17. The method of claim 11, wherein the mammalian subject is a human subject.

18-19. (canceled)

20. A method of producing an immune response against a drug of abuse in a mammalian subject, the method comprising administering the immunogenic formulation of claim 9 to the mammalian subject in an amount effective to produce an immune response in the mammalian subject against the drug of abuse.

21. A method of reducing the effect of a drug of abuse in a mammalian subject, the method comprising administering the immunogenic formulation of claim 9 to the mammalian subject in an amount effective to reduce the effect of the drug of abuse in the mammalian subject.

22. A method of treating drug addiction in a mammalian subject, the method comprising administering the immunogenic formulation of claim 9 to a mammalian subject addicted to a drug of abuse in an amount effective to reduce the addiction in the mammalian subject to the drug of abuse.
